(19) United States
(12) Patent Application Publication
(10) Pub. No.: US 2007/0213687 A1
(43) Pub. Date: Sep. 13, 2007

(54) Cannulation device with segmented tip

(76) Inventor: David E. Barlow, Coopersburg, PA (US)

Correspondence Address: SCULLY SCOTT MURPHY & PRESSER, PC, 400 GARDEN CITY PLAZA, SUITE 300, GARDEN CITY, NY, 11530, US

(21) Appl. No.: 11/715,098

(22) Filed: Mar. 7, 2007

Related U.S. Application Data
(60) Provisional application No. 60/779,778, filed on Mar. 7, 2006.

(51) Int. Cl. A61M 25/00

(52) U.S. Cl. 604/523

(57) ABSTRACT

A cannulation device is provided that includes: an elongated flexible sheath having a lumen and a distal end; a segmented tip including one or more segmental elements disposed at the distal end; a control member disposed in the lumen and fixed to a distal most segmental element of the one or more segmental elements; and an actuator for selectively tensioning and relaxing the control member to decrease and increase, respectively, a flexibility of the segmented tip.

### CROSS-REFERENCE TO RELATED APPLICATIONS

This application is based on and claims the benefit of priority from U.S. Provisional Application Ser. No. 60/779,778 filed on Mar. 7, 2006, the entire contents of which is incorporated herein by reference.

### FIELD OF THE INVENTION

The present invention relates to methods and apparatus for improving the functionality of various guidewires, cannulas and other types of cannulation devices for entering and traversing small anatomic lumens within the human body.

### BACKGROUND OF THE INVENTION

Various diagnostic and therapeutic medical procedures require the insertion of guidewires, tubes, cannulas and other slender devices into orifices, lumens and internal pathways of the human body. A variety of designs are available to facilitate passage of these devices into the desired orifice or into the desired segment of a bifurcating lumen. Guidewires with spiral-wound tips of various flexibilities, guidewires with removable core wires, catheters with tapered distal tips, and catheters with controllable deflection are all attempts at improving the cannulation ability of these types of devices.

In many applications of medicine it is required that a guidewire or tube be placed into an anatomical orifice, or inserted into a specific anatomical lumen. A guidewire thus placed may then serve as a means for guiding other devices into the same lumen. A guidewire thus placed is also useful for maintaining an access tract into the body when one medical device is removed and exchanged for another medical device, and follows the same pathway into the body.

Cannulas with lumens thus placed in the body are useful for injecting drugs, x-ray contrast solutions, and other agents into various organs and ductal systems within the body. In addition, the lumen of cannulas are used to guide various therapeutic devices into position, such as radiofrequency cutting devices, flexible needles, snares, etc.

Once a cannula is inserted into an anatomical lumen, it generally tracks in the direction of the lumen. However, placing such a device into the orifice of open lumens is frequently quite difficult. In particular, cannulating the Papilla of Vater (the opening into the duodenum of the pancreato-biliary system) is frequently one of the more difficult tasks in an endoscopic procedure referred to as ERCP (endoscopic retrograde cholangiopancreatography). Typically the Papilla of Vater is first cannulated using a thin plastic tube commonly referred to as an ERCP cannula. This cannula is first passed through the instrumentation channel of a duodenoscope, a flexible endoscope specifically designed for performing ERCP. The endoscopist than maneuvers the tip of the endoscope to position the distal tip of the cannula near the orifice of the central duct of the Papilla of Vater. A control mechanism incorporated in the duodenoscope, typically referred to as the “elevator”, enables the endoscopist to move the distal end of the cannula, which protrudes from the endoscope in order to position it precisely in the desired location on the surface of the Papilla of Vater. The endoscopist will repeatedly reposition the endoscope, adjust the position of the elevator, and move the cannula in and out of the endoscope as he/she probes the Papilla of Vater. The location of the lumen opening is often difficult to see precisely. It is sometimes located by a telltale droplet of bile oozing from the surface, or a small red spot on the surface of the papilla.

In addition to the requirement to place the cannula tip precisely against the often difficult-to-see luminal orifice, it is important that the lumen opening be probed in a direction parallel to its axis. If the access device is pressed against the lumen opening in line with its axis, the device will easily pass into the lumen. If the opening is probed at a significant angle with respect to the axis of the lumen, the device will likely impinge against the wall of the lumen and fail to enter the orifice. Since the direction of the axis of the lumen is not visible from outside the papilla, the endoscopist may hazard a guess regarding the direction that the lumen takes, but he/she cannot know for sure until the lumen is sufficiently cannulated to allow for the injection of contrast media into the lumen for observation via fluoroscopy.

In addition to difficulties in ascertaining the location and direction of the orifice and luminal tract, it is also frequently difficult for the endoscopist to maneuver the endoscope into position to allow the cannulation device to enter the papilla along the axis of the lumen.

As a result of all of these difficulties, endoscopists may blindly probe the papilla, hoping that the cannula falls into the opening; and if unsuccessful, reposition the equipment and blindly probe again. This activity may traumatize the papilla, causing it to become inflamed and even more difficult to cannulate.

Because of the difficulty in positioning the cannulation device to easily enter the orifice, many prior art attempts have been made to improve the cannulation ability of these devices. Some commercialized ERCP cannulas have a tapered tip in order to reduce the diameter of the most distal part that first enters the orifice. Other designs have rounded or hemispherically shaped tips that are reportedly easier to insert than cannulas with flattened or blunted tips. Most cannulas are made of plastic, and the inherent flexibility of the device is determined by the choice of polymer(s) used to make the cannula, together with the diameter and thickness of the cannula wall. While attempts are made to make these devices flexible, in fact, the distal tip of current cannulation devices are too rigid to allow them to make an abrupt turn to enter a lumen if probed off-axis.

A similar problem occurs when a cannula is further inserted into a ductal lumen and encounters a bifurcation in the lumen. In order to easily enter the selected bifurcation, the cannula must often make an abrupt turn in one direction or another. This situation is encountered when cannulating the pancreatobiliary tree, urinary tract, and more frequently when cannulating cardiac and peripheral blood vessels. To enable cannulation devices such as guidewires to enter a selectable arm of the bifurcation, cannulation devices are often designed to allow their distal tip to be selectively stiffened or deflected. One means of doing this is to employ a guidewire with a central lumen through which a core wire is advanced. When this core wire is within the guidewire coils, the guidewire is relatively stiff. When the core wire is withdrawn, the guidewire tip becomes more flexible. However, these devices have only two inherent degrees of flexibility, the flexibility that exists when the core wire is in place and the flexibility that exists when the core wire is removed. Furthermore, this design does not enable the wire to become most flexible were it is most needed, that is at its very distal-most end.

### SUMMARY OF THE INVENTION

Medical apparatus is provided herein comprising a guide wire or lumened catheter having a segmented tip, the flexibility of which is controllable by the operator. The ability to soften or stiffen, that is to make the tip more flexible or more rigid, by incorporating segmental elements over a control wire enables the medical apparatus to cannulate ductal orifices or pass into selected bifurcating lumens with greater ease and greater control than existing devices.

A methodology of cannulation using such medical apparatus allows the very tip of the cannulation device to become extremely flexible. Furthermore, the device can be adjusted by the operator to exhibit a preset amount of flexibility; or the operator can change the device's tip flexibility during use to further facilitate cannulation activity. In the medical apparatus, a series of repeating beads or segments are held onto the tip of the cannulation device by means of a centrally located control wire. When the control wire is extended (loosened), the segments separate and the flexibility of the segmented part is primarily dependant upon the inherent flexibility of the control wire. In this condition the flexibility of the device can be made quite soft, such as by using a polymer, or very thin metal control wire. When the handle of the device is operated to put tension on the control wire, the segments are compressed and the entire shaft of the device is placed under tension. This causes the segmented part of the cannulation device to become more rigid. The amount of additional rigidity is dependant upon the degree of tension that the operator places on the control wire.

The medical apparatus may have several embodiments. In one embodiment, the amount of tension in the control wire, and therefore the degree of flexibility in the tip of the device is preset by the manufacturer by fixing the distal and proximal ends of the control wire with a preset amount of tension. Another embodiment of the device allows the tension in the control wire to be set by the operator via a spring device and/or a screw device that puts a prescribed amount of tension in the cannulation device as it is being used. Another embodiment of the device allows the operator to customize the flexibility of the device to his particular preference or application by altering the amount of tension in the control wire by means of the handle of the device. A further embodiment of the device enables the operator to quickly change the amount of tension in the control wire from a preset minimum amount of tension to a preset maximum amount of tension by means of a handle that has appropriate stops and adjustments built into the handle.

Depending on the physical size of the cannulation device, the medical apparatus can serve either the function of a guidewire, or the function of a cannula tube. If the device is made with a relatively small outer shaft diameter, the device can be inserted into a body lumen as an access device, over which another lumened device is slid into place. If the device is made with a relatively larger diameter and incorporates a lumen sufficient for the injection of fluids, the device can be used as a cannula tube for carrying fluids from the handle end of the device into the cannulated lumen within the body. In this embodiment of the present invention, fluids such as x-ray contrast media can be injected through the length of the cannulation device, exiting at the segmented tip.

Several additional embodiments of the medical apparatus are envisioned. The shaft of the device can be constructed of coiled metal wire (that is, a coil-tube construction), or alternatively of polymer tubing, or both. The profile of the various segments at the tip can have various shapes such beads that are spherical, beads that are generally cylindrical, or beads that have a variety of profiles. Furthermore, the segments can be of uniform size, or could be tapered with the most distal of the segments being smaller in diameter to facilitate insertion into small openings.

Additionally the number of segments at the distal tip can be varied to suit the particular intended application.

Thus, an object of the medical apparatus is to provide a method and apparatus for entering a ductal orifice with greater control and ease.

Another object of the medical apparatus is to provide a cannulation device with a segmented tip, which has a preset degree of flexibility.

A further object of the medical apparatus is to provide a cannulation device with a segmented tip with an operator-controlled variable degree of flexibility.

Yet another object of the medical apparatus is to provide a cannulation device with a segmented tip, which allows for the injection of fluid through the cannulation device.

Yet another object of the medical apparatus is to provide a cannulation device with a single moveable element at its tip that enables easy cannulation and allows for the injection of fluid through the cannulation device.

These and other objects of the present invention will be apparent from the drawings and detailed description herein.

Accordingly, a cannulation device is provided. The cannulation device comprising: an elongated flexible sheath having a lumen and a distal end; a segmented tip including one or more segmental elements disposed at the distal end; a control member disposed in the lumen and fixed to a distal most segmental element of the one or more segmental elements; and an actuator for selectively tensioning and relaxing the control member to decrease and increase, respectively, a flexibility of the segmented tip.

The distal end of the sheath can be one of tapered and rounded.

The one or more segmental elements can have a spherical shape.

The one or more segmental elements can have at least a curved surface portion.

The one or more segmental elements can comprise a plurality of segmental elements, where the control member can be fixed to the distal most of the plurality of segmented elements and the other of the plurality of segmented elements can have a lumen for disposal of the control member therethrough. The distal most segmented element can have a diameter less than the other of the plurality of segmented elements. Each of the other of the plurality of segmented elements can have a diameter greater than an adjacent segmented element in a distal direction.

The actuator can comprise: a body connected to a proximal end of the sheath; and a movable member movable relative to the body; wherein the control member can be fixed to the movable member such that relative movement of the control member and the body selectively tensions and relaxes the control member to decrease and increase, respectively, the flexibility of the segmented tip. The body can further comprise a mechanism for limiting the movement of the movable member. The mechanism for limiting the movement of the movable body can comprise: a stopper disposed on the control member and having an interfering portion; a distal stop member disposed on the body for engaging the interfering portion when the movable member is moved in a distal direction; and a proximal stop member disposed on the body for engaging the interfering portion when the movable member is moved in a proximal direction. At least one of the distal stop member and proximal stop member can be adjustably disposed on the body. At least one of the distal stop member and proximal stop member can include a shape for allowing the interfering portion to pass through without engagement.

The cannulation device can further comprise a spring element for applying a predetermined tension to the control member. In which case, the cannulation device can further comprise an adjustment member disposed on the body for varying an amount of the predetermined tension by the spring element.

Where the actuator includes the body and movable member, the cannulation device can further comprise a spring element for biasing the movable member in one of a distal or proximal direction. In which case, the cannulation device can further comprise an adjustment member disposed on the body for varying an amount of bias by the spring element.

Also provided is a method for cannulating one of a ductal orifice within a body and selectively cannulating a bifurcating lumen within a body. The method comprising: introducing a cannulation device to a site of cannulation; providing the cannulation device with a segmented tip including one or more segmental elements disposed at a distal end; and selectively adjusting a flexibility of the segmented tip of the cannulation device to facilitate entry of the device into the ductal orifice.

The ductal orifice can be the Papilla of Vater.

The site of cannulation can be a bifurcation in one of a duct or vessel.

The introducing can comprise introducing the cannulation device through an instrumentation channel of an endoscope, the instrumentation channel having an exit port positioned adjacent the entrance.

### BRIEF DESCRIPTION OF THE DRAWINGS

FIG. 1 is lateral sectional schematic view of a portion of the human upper digestive tract illustrating a flexible endoscope in a typical position for cannulating the Papilla of Vater.

FIG. 2 is a magnified view of a portion of the anatomy shown in FIG. 1 illustrating how the elevator within the distal tip of the endoscope helps to deflect the cannula into the proper shape and position for cannulating the Papilla of Vater.

FIG. 3 is a cross-sectional schematic view of a prior art ERCP cannula pressing against the ductal opening of the Papilla of Vater without sliding into the lumen.

FIG. 4A is a schematic view of a first embodiment of the cannulation device of the present invention deflecting to enter the ductal opening of the Papilla of Vater.

FIG. 4B illustrates further advancement of the cannulation device shown in FIG. 4A into the central duct of the Papilla of Vater.

FIG. 5 is a longitudinal sectional view of the distal tip of the first embodiment of the cannulation device.

FIG. 6A is a longitudinal view of the distal tip of the first embodiment of the cannulation device illustrating that the segmented tip becomes more flexible when tension in the control wire is released.

FIG. 6B is a longitudinal view of the distal tip of the first embodiment of the cannulation device illustrating that the segmented tip becomes straight and more when tension in the control wire is increased.

FIG. 7 is an external view of the first embodiment of the cannulation device providing an overall view of the handle, the shaft and the segmented tip of the device.

FIG. 8 is a longitudinal cross-sectional view of the distal tip of the first embodiment of the cannulation device illustrating how advancement of the control wire moves the segmental elements away from the tip of the sheath allowing fluid to flow out of the lumen of the device.

FIG. 9 illustrates a second embodiment of the handle of the cannulation device, illustrating how movement of the slider and control wire is limited by stop nuts.

FIG. 9A is an external view of the stopper and control wire components illustrated in FIG. 9.

FIG. 9B is a sectional view of the handle of the second embodiment taken along line A-A in FIG. 9. This figure illustrates how the rotational position of the stop nut will either limit passage of the stopper or allow it to pass freely.

FIG. 10 is an external view of a third embodiment of the handle of the cannulation device of the present invention. This figure illustrates how alternative embodiments of the handle can accomplish the same functions of the embodiment illustrated in FIG. 9.

FIG. 11 is an external view of a fourth embodiment of the handle for the cannulation device of the present invention.

FIG. 12 is an external view of a fifth embodiment of the handle for the cannulation device of the present invention.

FIGS. 13A-13G show various embodiments of the distal end of the cannulation device, illustrating some of many possible variations in the design of the segmental elements.

FIGS. 14A-14C are longitudinal cross-sectional views of various types of distal tips found on prior art cannulas.

FIG. 14D is a longitudinal cross-section of the distal end of an alternative embodiment of the present invention illustrating a single moveable element at the tip of the cannulation device.

FIG. 14E illustrates that when the element occluding the tip of the cannulation device illustrated in FIG. 14D is separated from the tip of the sheath it will allow fluid to flow through the sheath.

FIG. 14F is a longitudinal cross-section of the distal end of another alternative embodiment of the present invention.

FIG. 14G is a longitudinal cross-section of the distal end of another alternative embodiment of the present invention.

FIG. 14H is a longitudinal cross-section of the distal end of another alternative embodiment of the present invention.

FIG. 15 is an external view of one embodiment of a cannulation device with a single moveable element at its distal tip.

FIG. 16 is a schematic drawing illustrating how a cannulation device of the present invention has application in selective cannulation of bifurcating ducts and vessels.

### DETAILED DESCRIPTION

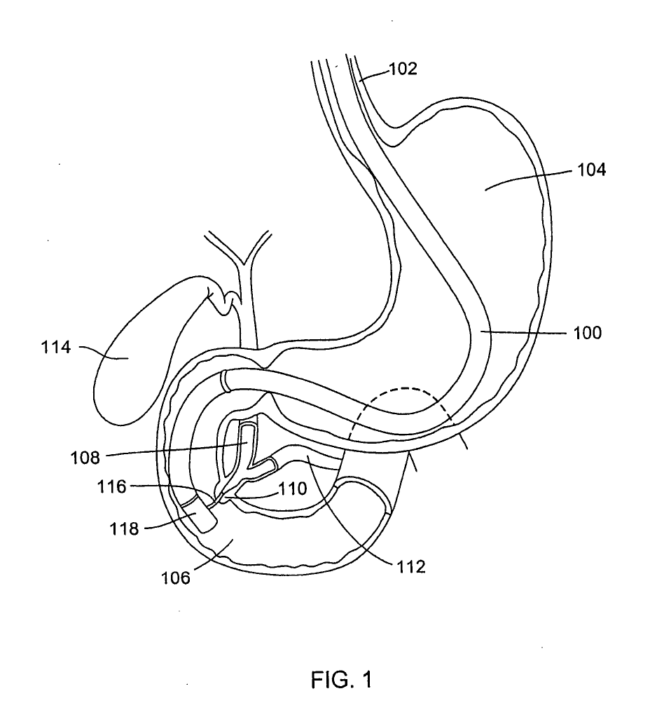

FIG. 1 illustrates the typical anatomical position of a flexible endoscope when used to cannulate the Papilla of Vater. A flexible endoscope 100, in this case a duodenoscope, is passed through the patient's mouth (not shown), the esophagus 102, the stomach 104, and into the first part of the duodenum 106. The endoscopist then maneuvers a distal end 118 of the endoscope so that it is positioned adjacent to the Papilla of Vater 110.

FIG. 1 also illustrates a portion of the pancreatobiliary system. The pancreatic duct 112 joins the common bile duct 108 coming from the gallbladder 114 and liver (not shown). Secretions from the liver and pancreas are collected by this ductal system and empty into the duodenum via a small orifice in the Papilla of Vater 110. One of the objectives of the medical procedure known as ERCP (endoscopic retrograde cholangiopancreatography) is to cannulate the Papilla of Vater with a thin flexible cannula 116 through which x-ray contrast media is injected to enable the pancreatobiliary ductal system to be seen radiographically via a fluoroscope.

FIG. 2 shows an enlarged view of the distal end of the endoscope and the cannulation procedure. The distal end 118 of the endoscope 100 with side-viewing optics is positioned in the duodenum 106 with its tip close to the Papilla of Vater 110. The operator can move an elevator 200 contained in the endoscope tip up and down relative to the endoscope by means of an elevator control wire 206 which runs up the shaft of the endoscope from the elevator to the control handle of the endoscope (not shown). The operator observes the position of the catheter relative to the Papilla of Vater as he/she manipulates the position of the endoscope itself and the cannula passing through the endoscope. The line of sight (direction of view) 208 of the endoscope is approximately perpendicular to the axis of the endoscope to enable it to optimally visualize the Papilla of Vater en face. With difficult but careful manipulation of the endoscope and the cannula, the operator is generally able to insert the tip of the ERCP cannula 116 through the orifice of the papilla, and into the lumen 202 of the ductal system.

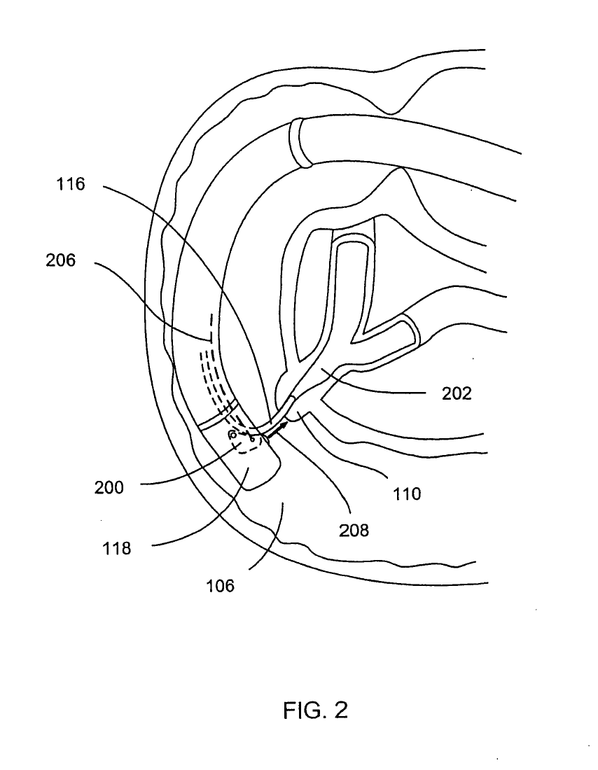

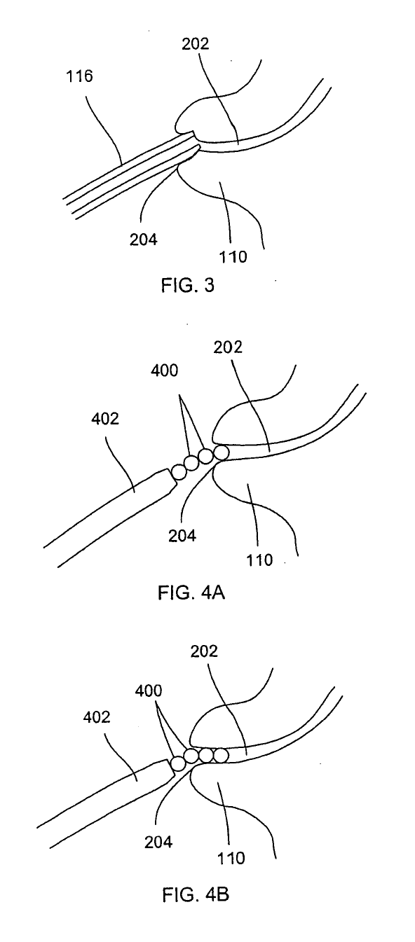

FIG. 3 illustrates a problem that is commonly encountered when a standard plastic ERCP cannula 116 is inserted into the ductal orifice 204 of the Papilla of Vater 110 at an angle with the duct 202. Pushing the tip of the cannula against the papilla will often embed it in the tissue rather than allowing it to slide successfully into the duct.

FIG. 4A illustrates the advantage of a first embodiment. The distal end of the cannulation device 402 is composed of a series of segments 400 which allow it to flex and align with the axis of the ductal lumen 202. As FIG. 4B illustrates, once the most distal few segments enter the lumen, they guide the rest of the cannulation device 402 into the duct. The flexibility of the segmented tip prevents it from embedding itself in the wall of the tissue, and enables it to preferentially slide into the duct itself.

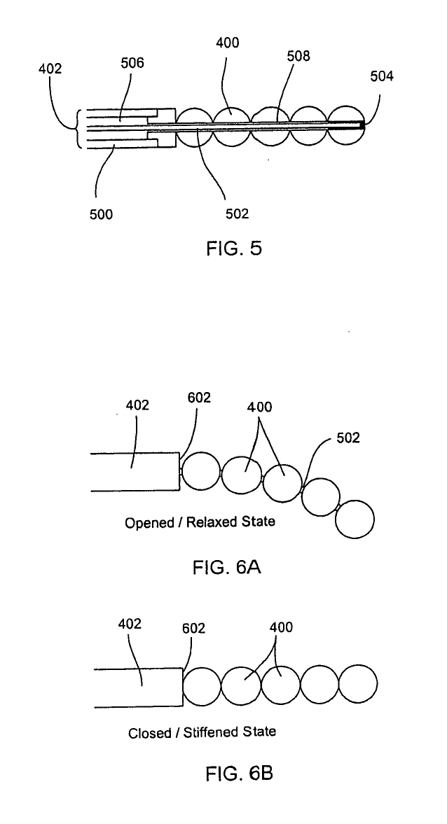

FIG. 5 is a cross-sectional view of the tip of the first embodiment shown in FIGS. 4A and 4B. The shaft of the cannulation device 402 is composed of two main components. An outer component is a sheath 500 composed of either plastic tubing or wound wire (coil wire) that creates the outer surface of the device. Inside this sheath is a control wire 502 that runs throughout the length of the sheath's lumen 506 and terminates by running through the center of a series of segmental elements 400. In the embodiment illustrated in FIG. 5, these segmental elements take the form of spherical beads each of which has a central lumen 508 through which the flexible control wire 502 passes. The dimension of the control wire and the dimension of the holes within the beads are such that the beads are loosely strung over the control wire. The control wire is firmly affixed to the most distal of the beads at a fixation point 504. Depending upon the materials selected for the control wire and the beads, the method of fixation may be soldering, an epoxy adhesive, thermoplastic welding, etc. While FIG. 5 illustrates an embodiment with five beads, any number of segments could be used, depending upon the desired tip characteristics and the particular intended application for the device.

FIGS. 6A & 6B illustrate how the shape and flexibility of the segmented tip will change depending upon the position and tension placed on the control wire. Since the distal-most bead is firmly affixed to the flexible control wire, and since extension of the control wire from the tip of the shaft 602 will cause the beads to separate—releasing the tension in the control wire and extending the beads will cause the beaded tip to become increasingly more flexible. The final rigidity of the beaded section will be largely determined by the inherent flexibility of the control wire 502 itself. On the other hand, if tension is put on the control wire to pull the beads against one another and against the distal end 602 of the shaft 402, the beaded portion of the cannulation device will become increasingly more rigid and less easily deflected, as illustrated in FIG. 6B. By varying the amount of tension placed on the control wire, the tip of the cannulation device can vary progressively from being very floppy to becoming very rigid. This ability to immediately change the flexibility of the tip of the device over a wide range is part of the utility of the apparatus.

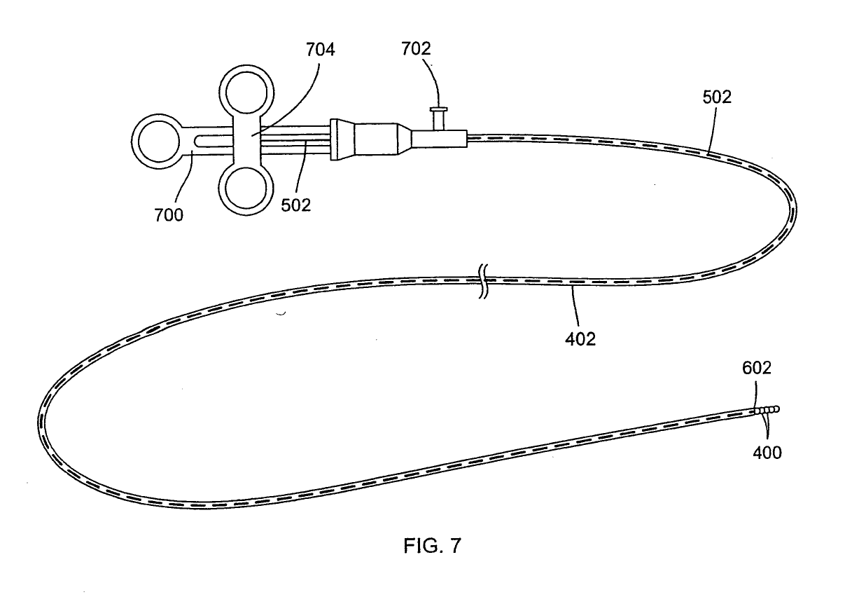

FIG. 7 illustrates the components of a complete cannulation device of the first embodiment. The shaft of the device 402 has sufficient length to reach the target anatomy of the patient. In the ERCP application illustrated in FIG. 1, the shaft of the device must be at least as long as the channel of the endoscope through which it passes, plus some additional length so that it can be handled conveniently. In other applications the catheter may be inserted into the body directly without passing through another medical instrument, may be inserted through an access cannula (e.g., through a vascular catheter), or other appropriate means of entry into the body. The overall length of the device must be appropriate for its application. Likewise, the overall diameter of the shaft 402 and the diameter of the segmental elements 400 at its tip are made appropriate for the diameter of the duct or vessel that the device is intended to cannulate.

The segmental elements 400 at the distal tip of the device are loosely strung over a flexible control wire 502 that runs through the lumen of the shaft to a handle 700 operated by the user. The diameter of the control wire and the materials of its construction are chosen to match the characteristics of the device with its intended application. The control wire may be composed of metal which would cause it to have more spring when in a relaxed (non-tensioned state), or be composed of a polymer which would cause it to be more floppy when relaxed, or composed of other material(s) exhibiting characteristics desirable for the particular intended medical application.

The level of tension in the control wire 502, and thereby the degree of rigidity in the tip of the device, is controlled by the operator by moving the slider 704 relative to the handle 700. The handle has no stops or adjustments, allowing the operator to fully control the amount of tension placed on the handle, and thereby the degree of rigidity in the segmented tip.

The embodiment illustrated in FIG. 7 also contains an injection port 702 on the handle of the device, through which the operator can inject fluids (such as x-ray contrast media) through the lumen of the catheter 402, which fluid then exits the distal end of the catheter 602.

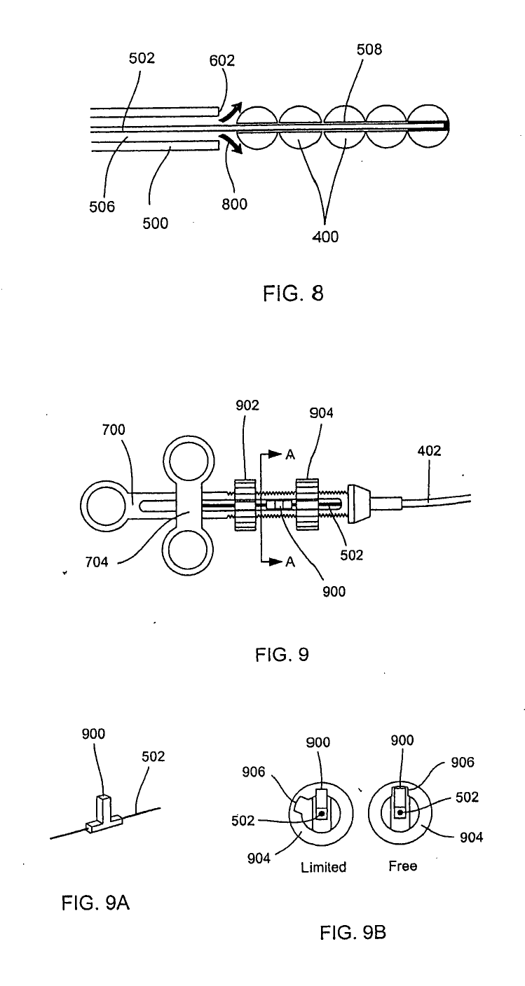

FIG. 8 illustrates that to allow fluid injected into the lumen 506 of the sheath 500 to exit from the tip 602 of the sheath, the control wire 502 must be extended to create a gap between the most proximal of the segmental elements 400 and the tip of the sheath 602. This will allow fluid to flow 800 from the cannulation device into the duct in which it is residing.

FIG. 9 illustrates a second embodiment of an appropriate handle 700 for the cannulation device. Similar to the previous embodiment, the control wire 502 is connected to the slider 704. Movement of the slider puts more or less tension in the control wire, thereby changing the flexibility of the segmented tip (not shown). However, in this embodiment, a stopper 900 is affixed to the control wire 502 as illustrated in FIG. 9A. This stopper limits the movement of the control wire. A proximal stop nut 902 limits movement of the control wire as the slider on the handle is “closed”, thereby limiting the maximum amount of tension placed on the segmental elements and the maximum rigidity of this section of the cannulation device. A distal stop nut 904 conversely limits the amount that the handle can be “opened”, thereby limiting the distance that the control wire can be extended from the tip of the cannula. In the embodiment illustrated in FIG. 9, the position of these maximum and minimum settings can be changed by the operator by changing the position of the threaded stop nuts on the handle. Furthermore, each stop nut has a cutout 906 which allows the stopper 900 to bypass the stop nut when the stop nut is rotated into the “free” position, as illustrated in FIG. 9B. This feature allows the operator to quickly put the handle into a configuration in which the maximum and minimum limits of the control wire movement are overridden. When the stop nuts are put into the “free” configuration, the slider 704 can move back and forth freely without the stopper 900 encountering the stop nuts (902 & 904).

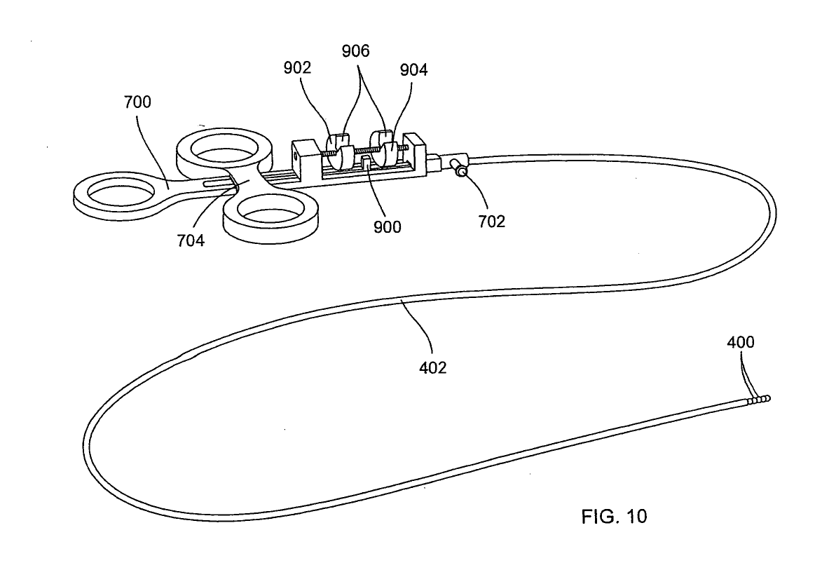

FIG. 10 illustrates a third embodiment of the handle 700 of the cannulation device of the present invention. As in the handle illustrated in FIG. 9, movement of the slider 704 moves the control wire and changes the rigidity of the segmented tip. However, movement of the control wire is limited by a stopper 900 affixed to the control wire. This stopper limits the movement of the control wire to the positions set by the proximal 902 and distal 904 stop nuts. The position of these stop nuts can be changed by the operator by rotating them over the threaded shaft on which they are mounted. Furthermore, each of the stop nuts has a cutout 906 in it. The operator can quickly override the limits of the stop nuts by rotating the nuts to place them in a configuration whereby the stopper 900 passes through the cutouts 906 and is no longer limited by the stop nuts.

The embodiment illustrated in FIG. 10 also contains an injection port 702 through which fluids may be injected into the cannulation device.

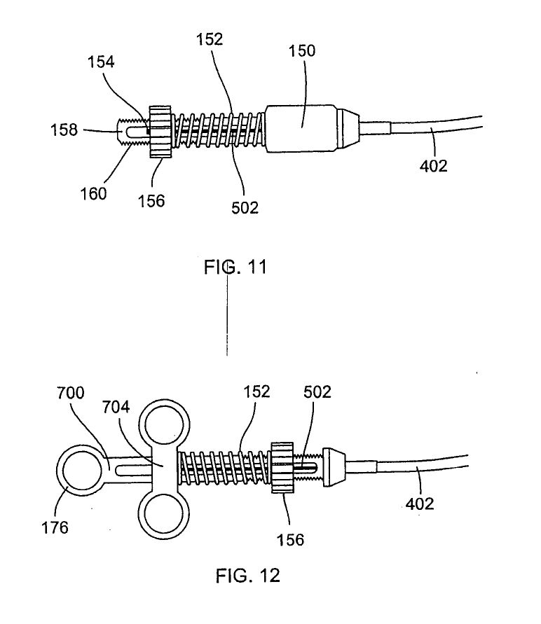

Unlike the handles illustrated in FIGS. 7, 9 & 10 which require that the operator constantly operate and monitor the tension in the control wire, a fourth embodiment of the handle which is illustrated in FIG. 11 will apply a constant, and in this embodiment, an adjustable amount of tension in the control wire. The handle 150 is attached to the proximal end of the shaft 402 of the cannulation device. The proximal end of the control wire 502 running through the length of the shaft is rotatably fixed 154 to a stiffness adjustment nut 156 threaded 160 on the shaft 158 of the handle. As this adjustment nut 156 is screwed away from the handle 150 it pulls on the control wire 502 with an increasing amount of tension, making the segmented tip of the device more rigid. When the adjustment nut is screwed in the opposite direction it releases the tension in the control wire, reducing the rigidity of the segmented tip. A spring 152 on the handle puts pressure on the stiffness adjustment nut 156 thereby maintaining it in the position set by the operator. This embodiment of the handle allows the operator to set a constant amount of tension in the control wire, and thus a constant degree of rigidity in the segmented tip without the need to constantly hold (or operate) the handle. The degree of rigidity in the segmented tip can be changed by the operator as needed during the procedure.

FIG. 12 illustrates a fifth embodiment of the handle of the cannulation device. In this embodiment the control wire 502 is connected to a slider 704 on the handle 700. A spring 152 places a constant, preset amount of force on the slider, and thereby a constant, preset amount of tension in the control wire 502 which in turn produces a constant preset degree of rigidity in the segmented tip. This preset amount of rigidity in the tip of the device can be adjusted by changing the position of the stiffness adjustment nut 156. In this embodiment, the operator has the ability to further enhance the rigidity of the tip of the device by “closing” the handle by pulling the slider 704 towards the thumb ring 176. This action increases the tension in the control wire, stiffening the tip. In a similar manner, the operator can “open” the handle by moving the slider 704 away from the thumb ring 176. This action compresses the spring 152, relaxing the tension in the control wire and making the segmented tip more flexible.

Although several embodiments for the handle of the cannulation device have been illustrated, one of ordinary skill in the art, in light of this teaching, can generate additional embodiments and modifications without departing from the spirit or exceeding the scope of the claimed invention.

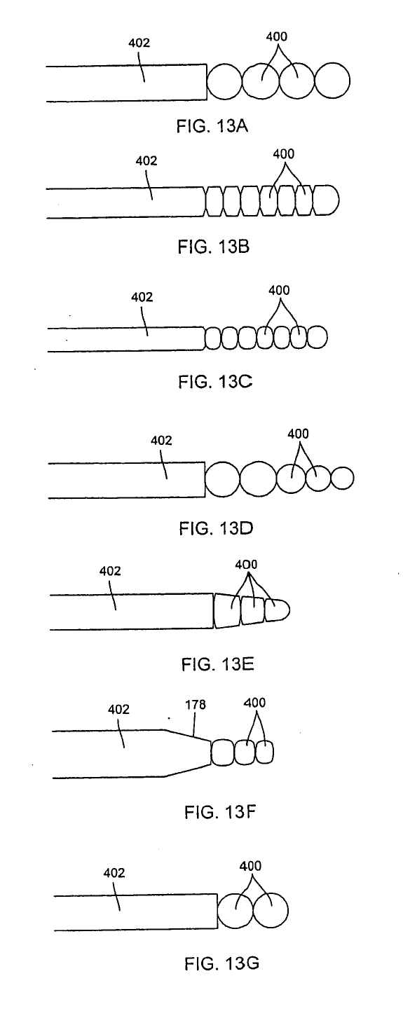

FIG. 13 illustrates that the segmental elements 400 at the distal tip of the catheter 402 can likewise have several embodiments. As illustrated in FIG. 13A, in a first embodiment of the tip, the segmental elements have an approximate spherical shape. Alternatively, as illustrated in FIG. 13B, the segmental elements may have an approximate cylindrical outermost surface with ends that allow them to flex with respect to each other. As FIG. 13C illustrates, the segmental elements may have other alternative shapes that facilitate smooth entry in ductal orifices and smooth passage along the wall of the duct or the vessel it is in. As FIGS. 13D & 13E illustrate, there is a benefit to tapering the size of the individual segmental elements. The small diameter of the distal-most element facilitates its entry into small orifices, while the increasing diameter of subsequent elements guides the larger diameter catheter into the ductal system. In addition to tapering the size of the segmental elements, the cannula sheath 402 may have a tapered distal tip 178, as illustrated in FIG. 13F.

The present invention does not specify or limit the number of segmental elements used in the cannulation device. The device may only two (FIG. 13G) to improve the control that the operator has over the tip of the device, or may have many elements to increase the radius of tip deflection depending on the specific application for which the cannula is designed.

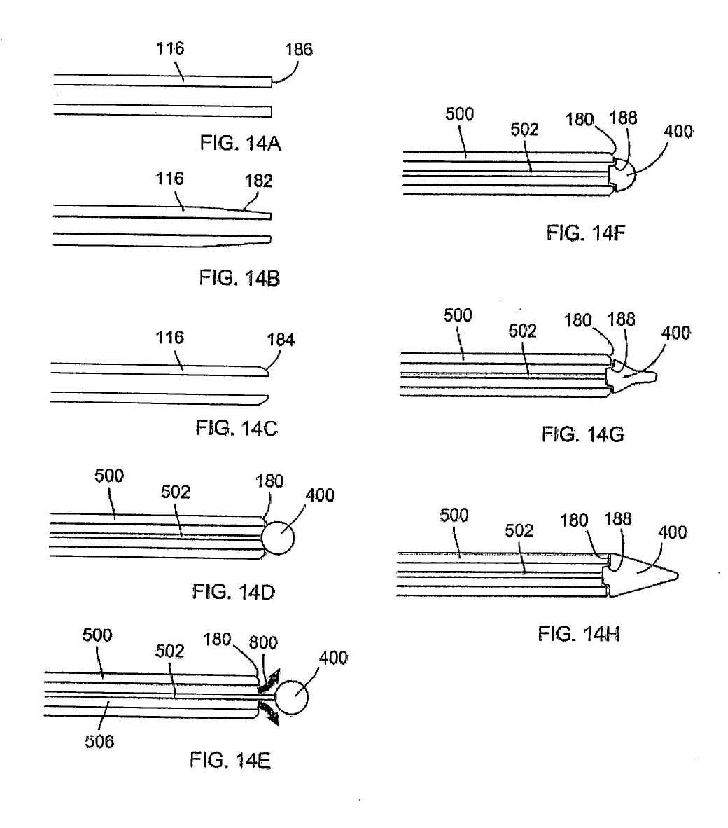

The tips of prior art cannulas 116 also have various configurations. Some are simply blunt tipped 186 tubes, as illustrated in FIG. 14A. Others have a tapered 182 distal end to improve entry into small diameter orifices, as illustrated in FIG. 14B. Some have a rounded 184 “nose”, as illustrated in FIG. 14C.

Another embodiment of the present invention, as illustrated by FIG. 14D, is to limit the number of segmental elements to one, and to configure the device such that when the element 400 is pulled against the sheath 500 by the control wire 502 running through its lumen, the combined profile of the element 400 and the distal tip of the cannula 180 create a surface that cannulates more easily than the open ended cannulas of prior art. The reason for this improved cannulation ability is that prior art cannulas are of necessity blunt at the very tip due to their open lumen. However, when the element of the current invention fills this open lumen with a rounded shape, it facilitates passage of the cannula tip over tissue and facilitates its entry into ductal orifices. As FIG. 14E illustrates, after cannulation, the control wire 502 is advanced moving the element 400 away from the distal end 180 of the catheter, allowing fluid to flow from the lumen 506 of the catheter out of the distal tip. This flow of fluid 800 allows the cannulation device to be used as a catheter following ductal cannulation.

As FIG. 14F, FIG. 14G and FIG. 14H illustrate, the shape of the single element 400 may have many embodiments, including a proximal surface 188 that serves to position the element 400 in the center of the catheter's distal end 180.

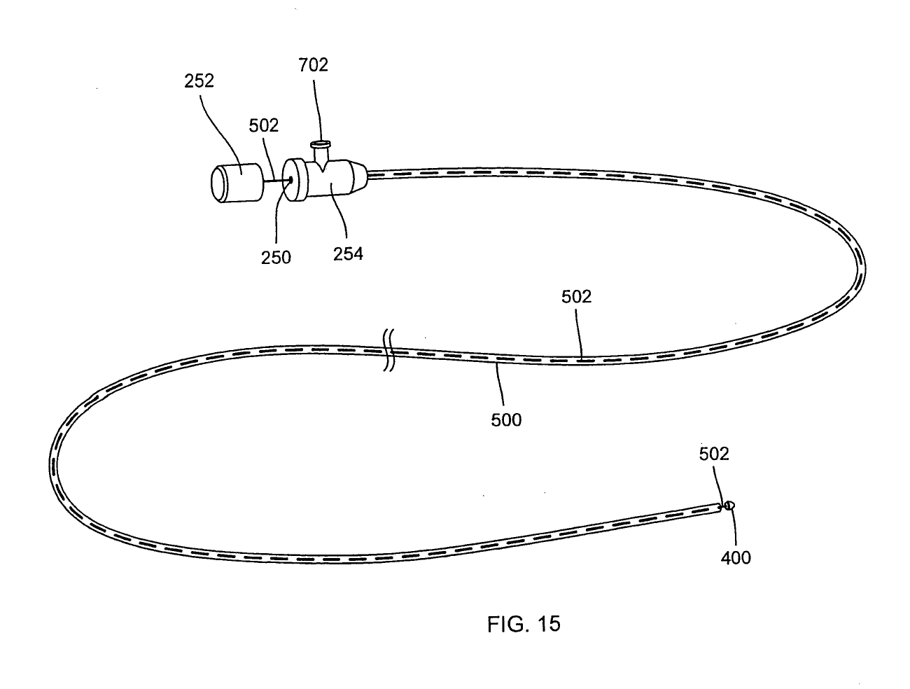

FIG. 15 illustrates the components of one embodiment of a cannulation device with a single element at its distal tip. The element 400 is affixed to the distal end of a control wire 502 that runs through the catheter sheath 500, through a control body 254, and to a proximal handle 252. As the handle 252 is moved towards and away from the control body 254, it moves the distal element 400 away from and towards the distal end of the sheath, respectively. An injection port 702 on the control body allows the injection of fluid down the open lumen of the sheath, exiting at its distal end. A fluid seal 250 at the proximal end of the control body prevents this fluid from leaking out around the proximal end of the control wire.

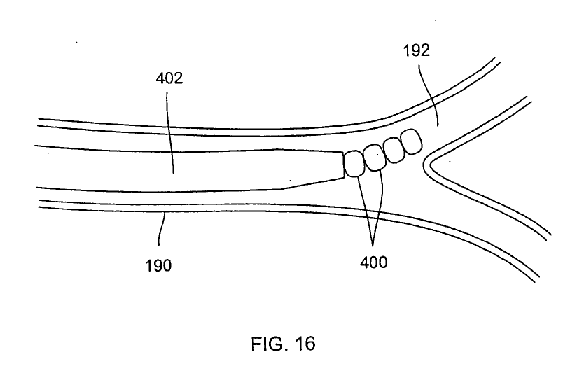

While the previous discussion has focused on the ability of the apparatus to enter a ductal orifice, the same is also useful in guiding passage of a cannula or guidewire through ductal systems such the Fallopian tubes, the urinary tract, biliary and pancreatic ducts, blood vessels and similar lumened anatomy. As FIG. 16 illustrates, the apparatus disclosed herein are useful for guiding the tip of commonly used guidewires and catheters into selected bifurcating tracts 192 of these ductal and vessel systems 190.

While there has been shown and described what is considered to be preferred embodiments of the invention, it will, of course, be understood that various modifications and changes in form or detail could readily be made without departing from the spirit of the invention. It is therefore intended that the invention be not limited to the exact forms described and illustrated, but should be constructed to cover all modifications that may fall within the scope of the appended claims.

What is claimed is:

1. A cannulation device comprising:
an elongated flexible sheath having a lumen and a distal end;
a segmented tip including one or more segmental elements disposed at the distal end;
a control member disposed in the lumen and fixed to a distal most segmental element of the one or more segmental elements; and
an actuator for selectively tensioning and relaxing the control member to decrease and increase, respectively, a flexibility of the segmented tip.

2. The cannulation device of claim 1, wherein the distal end of the sheath is one of tapered and rounded.

3. The cannulation device of claim 1, wherein the one or more segmental elements have a spherical shape.

4. The cannulation device of claim 1, wherein the one or more segmental elements have at least a curved surface portion.

5. The cannulation device of claim 1, wherein the one or more segmental elements comprise a plurality of segmental elements, the control member being fixed to the distal most of the plurality of segmented elements and the other of the plurality of segmented elements having a lumen for disposal of the control member therethrough.

6. The cannulation device of claim 5, wherein the distal most segmented element has a diameter less than the other of the plurality of segmented elements.

7. The cannulation device of claim 6, wherein each of the other of the plurality of segmented elements has a diameter greater than an adjacent segmented element in a distal direction.

8. The cannulation device of claim 1, wherein the actuator comprises:
a body connected to a proximal end of the sheath; and
a movable member movable relative to the body;
wherein the control member is fixed to the movable member such that relative movement of the control member and the body selectively tensions and relaxes the control member to decrease and increase, respectively, the flexibility of the segmented tip.

9. The cannulation device of claim 8, wherein the body further comprises a mechanism for limiting the movement of the movable member.

10. The cannulation device of claim 9, wherein the mechanism for limiting the movement of the movable body comprises:
a stopper disposed on the control member and having an interfering portion; and
a distal stop member disposed on the body for engaging the interfering portion when the movable member is moved in a distal direction.

11. The cannulation device of claim 9, wherein the mechanism for limiting the movement of the movable body comprises:
a stopper disposed on the control member and having an interfering portion; and
a proximal stop member disposed on the body for engaging the interfering portion when the movable member is moved in a proximal direction.

12. The cannulation device of claim 10, wherein the distal stop member is adjustably disposed on the body.

13. The cannulation device of claim 11, wherein the proximal stop member is adjustably disposed on the body.

14. The cannulation device of claim 10, wherein the distal stop member includes means for allowing the interfering portion to pass without engagement.

15. The cannulation device of claim 11, wherein the proximal stop member includes means for allowing the interfering portion to pass without engagement.

16. The cannulation device of claim 1, further comprising a spring element for applying a predetermined tension to the control member.

17. The cannulation device of claim 16, further comprising an adjustment member disposed on the body for varying an amount of the predetermined tension by the spring element.

18. The cannulation device of claim 8, further comprising a spring element for biasing the movable member in one of a distal or proximal direction.

19. The cannulation device of claim 18, further comprising an adjustment member disposed on the body for varying an amount of bias by the spring element.

20. The cannulation device of claim 1, further comprising an injection port in fluid communication with the lumen of the flexible sheath to enable the injection of fluid from the injection port and through the lumen.

21. A method for cannulating one of a ductal orifice within a body and selectively cannulating a lumen within a body, the method comprising:
introducing a cannulation device to a site of cannulation;
providing the cannulation device with a segmented tip including one or more segmental elements disposed at a distal end; and
selectively adjusting a flexibility of the segmented tip of the cannulation device to facilitate entry of the device into the one of the ductal orifice and lumen.

22. The method of claim 21, wherein the ductal orifice is the Papilla of Vater.

23. The method of claim 21, wherein the cannulation site is a bifurcation in one of a duct or vessel.

24. The method of claim 21, wherein the introducing comprises introducing the cannulation device through an instrumentation channel of an endoscope, the instrumentation channel having an exit port positioned adjacent the ductal orifice.

25. The method of claim 21, further comprising injecting a fluid through a lumen in the cannulation device.

26. A cannulation device comprising:
an elongated flexible sheath having a lumen and a distal end;
a segmented tip including one or more segmental elements disposed at the distal end;
a control member disposed in the lumen and fixed to a distal most segmental element of the one or more segmental elements; and
tensioning means for providing a preset amount of tension in the control member to preset a flexibility of the segmented tip.

27. The cannulation device of claim 26, wherein the tensioning means comprises means for fixing distal and proximal ends of the control member with a preset amount of tension.
